(19) United States
(12) Patent Application Publication
(10) Pub. No.: US 2004/0014223 A1
(43) Pub. Date: Jan. 22, 2004

(54) Method intended for chemical and isotopic analysis and measurement on constituents carried by a drilling fluid

(76) Inventors: Annie Audibert, Croissy Sur Seine (FR); Alain Prinzhofer, Paris (FR)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/398,735

(22) PCT Filed: Oct. 2, 2001
(86) PCT No.: PCT/FR01/03033

(30) Foreign Application Priority Data
Oct. 10, 2000 (FR) 00/13037

(51) Int. Cl. G01N 33/24

(52) U.S. Cl. 436/30

(57) ABSTRACT

The present invention relates to a method intended for analysis and measurement on constituents carried by a well fluid during drilling operations, wherein the following stages are carried out:

taking a drilling fluid sample,

extracting the constituents in vapour form,

sending these constituents to a mass spectograph,

measuring the ¹³C/¹²C ratio of the isotopes of the carbon contained in the constituents.

### FIELD OF THE INVENTION

The present invention relates to a method intended for chemical and isotopic analysis of the carbons of liquid or gaseous constituents contained in a drilling fluid. Constituents are here understood to be hydrocarbons, for example from C1 to C8 including benzene, toluene, xylene, or gases such as CO₂. These constituents result from a drilling operation through formation layers, an operation which has the effect of breaking the rock while releasing the gases or the fluids contained in the rock pores. Drilling is conventionally performed with circulation of a fluid referred to as drilling fluid whose purpose is, among other things, to clean the drill bit and to bring the cuttings up to the surface. The constituents in question are therefore also carried up to the surface by means of this carrier. It is clear that, considering the flow rate of the drilling fluid in relation to the rate of destruction of the rock, the volume amount of said constituents is always relatively low in relation to the volume of mud.

### BACKGROUND OF THE INVENTION

There are well-known plants for carrying out qualitative and quantitative measurements on C1-C5 gas contained in a drilling fluid, these measurements (or mud logging) allowing to identify the geologic zones drilled for exploration, drilling and/or personnel safety reasons. Document FR-2,646,508 describes a method and a device allowing continuous sampling of the gaseous samples contained in a solids-containing liquid, notably a drilling fluid.

More recently, French patent application FR-99/12,032 filed by the applicant describes the conditions required to extract gaseous or liquid constituents contained in a drilling fluid, to convey these constituents in gaseous form, and to carry out analyses and measurements on these constituents. In order to be able to carry out correct analyses allowing to better determine the nature and the composition of the formations crossed by a borehole, the constituents must not condense in the elements of the system and the transit time of these constituents between the extraction point and the measurement point must be acceptable to allow the drilling operation to be monitored.

Document SU-483,645 describes taking a sample of a liquid in the borehole to obtain the geochemical carbon isotope and sulfur content in the zone surrounding the well. A comparison is then made between the composition of the drilling fluid filtrate that invades the formation and the composition of the fluids that saturate the formation, while following the methane isotopic composition criterion. The samples are taken straight through the layers studied so as to study their degree of saturation.

Several documents are aimed to determine the evolution of the composition of reservoirs from the wells by injection of tracer molecules (U.S. Pat. No. 5,892,147) or from the sediments (U.S. Pat. No. 5,388,456).

Although extraction of the light fraction of hydrocarbons (C1-C8) from mud is currently perfected on drilling platforms, no isotopic measuring device can be used on a drilling platform, in particular systems for coupling a gas chromatograph with an isotopic mass spectrometer by means of a combustion furnace converting each hydrocarbon to carbon dioxide (GC-C-IRMS), this type of device being designed today for use in a secure and air-conditioned laboratory (inventions by Crandall, Scalan and Hayes). The invention thus relates both to the coupling between a GC-C-IRMS and a system for hydrocarbon extraction from drilling fluids, and to the adaptation of this type of measuring device to field working conditions, where the fragile capillary tubes commonly used in the laboratory have to be replaced by protected catheters and where the electronics has to be displaced to protected premises far from the drilling site.

Continuous use of the isotopic signal for guiding a drilling operation can be made only with this invention, measurement of samples taken and analysed in the laboratory providing only a posteriori information which is useful only for later production stages, whereas continuous analysis during drilling allows better real-time guidance of the next stages of said drilling operation.

The present invention consists in analysing the drilling mud so as to obtain continuous data during drilling without any additional operation in the well, notably by means of downhole RFT type samplers.

### DETAILED DESCRIPTION

The present invention relates to a method intended for analysis and measurement on constituents carried by a well fluid during drilling operations, wherein the following stages are carried out:

taking a volume of a drilling fluid sample,

extracting the constituents in vapour form,

sending these constituents to a mass spectograph,

measuring the ¹³C/¹²C ratio of the isotopes of the carbon contained in said constituents.

In the method, the nature and/or the amount of the constituents can be determined by gas chromatography.

The invention also relates to a device intended for analysis and measurement on constituents carried by a well fluid during drilling operations. This device comprises a gas chromatograph coupled with a mass spectrometer suited to determine the ¹³C/¹²C ratio of the isotopes of the carbon contained in said constituents.

The precision of the isotopic measurements is higher than 1/1000.

By means of the method and of the device according to the invention, the drilling operation can be monitored from the continuous or discontinuous recording of the ¹³C/¹²C ratio. Correlating these ratio measurements with other mud logging type measurements allows to obtain a higher precision on the media crossed and on the constituents thereof.

The information obtained from chemical analyses on hydrocarbon gases proved interesting in the sphere of oil and gas exploration. During drilling, the chemical information (natures, amounts of hydrocarbon gases and proportions between hydrocarbons) is a potent guide for control of the hole localization, notably in the case of horizontal drilling, where it is thus possible to rapidly distinguish the proximity of the cap rock levels, the groundwater levels, or the potentially producing levels.

Combination of these chemical measurements with the measurement of the isotopes of the carbon contained in each hydrocarbon compound provides new pertient information during drilling:

the chemical signal of the gas (amounts of C1-C5 in the cuttings) can be highly disturbed by the generation of gas of bacterial origin. Combination of the chemical and isotopic measurements allows to disregard this bacterial noise by allowing to identify the origins. An amount of thermogenic gas associated with deep reservoirs can thus be deduced in a more sensitive and reliable way. This thus deconvoluted signal can allow to estimate, during drilling, the more or less close proximity of a reservoir filled with hydrocarbons,

a petroleum reservoir is generally crossed by permeability barriers that separate blocks to be specifically developed. The isotopic signal measured during drilling can allow to better determine these permeability barriers because they generally correspond to isotopic heterogeneities. The location, in the well casings, of perforations intended for production tests or for production proper can then be directly controlled by the results of these measurements,

the proximity of a water table in a petroleum reservoir increases the risk of biodegradation of the accumulated hydrocarbons, thus increasing the viscosity of the fluid which therefore becomes more difficult to produce. Since the water table can solubilize part of the gaseous hydrocarbons (mainly methane and ethane), and this dynamic solubilization being accompanied by a chemical and isotopic fractionation, continuous measurement of the isotopic signal must allow to detect the borehole approaching the water table and/or the hydrocarbons/water contact, whereas the chemical measurement alone shows a change only after the borehole has effectively crossed the hydrocarbons/water contact.

The present invention can be implemented by means of the system described in patent application FR-99/12,032 mentioned here by way of reference.

1) a method intended for analysis and measurement on constituents carried by a well fluid during drilling operations, characterized in that the following stages are carried out:
taking a volume of a drilling fluid sample,
extracting said constituents in vapour form,
sending these constituents to a mass spectograph,
measuring the ¹³C/¹²C ratio of the isotopes of the carbon contained in said constituents.

2) A method as claimed in claim 1, wherein the nature and/or the amount of said constituents is determined by gas chromatography.

3) A method as claimed in any one of the previous claims, wherein drilling is monitored by recording said ¹³C/¹²C ratio.

4) A device intended for analysis and measurement on constituents carried by a well fluid during drilling operations, characterized in that it comprises a gas chromatograph coupled with a mass spectrometer suited to determine the ¹³C/¹²C ratio of the isotopes of the carbon contained in said constituents.
